(19) United States
(12) Patent Application Publication
(10) Pub. No.: US 2001/0022960 A1
(43) Pub. Date: Sep. 20, 2001

(54) Hydrogen generating method and hydrogen generating apparatus

(75) Inventors: Yoshitsugu Kojima, Aichi-gun (JP); Kenichirou Suzuki, Aichi-gun (JP); Kazuhiro Fukumoto, Aichi-gun (JP); Megumi Sasaki, Aichi-gun (JP); Toshio Yamamoto, Aichi-gun (JP); Yasuaki Kawai, Aichi-gun (JP); Hiroaki Hayashi, Aichi-gun (JP)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: KABUSHIKI KAISHA TOYOTA CHUO KENKYUSHO, Aichi-gun (JP)

(21) Appl. No.: 09/758,194

(22) Filed: Jan. 12, 2001

(30) Foreign Application Priority Data
Jan. 12, 2000 (JP) P2000-003800
May 31, 2000 (JP) P2000-162897

(51) Int. Cl. C01B 3/08; A61L 9/00

(52) U.S. Cl. 423/657; 422/292

(57) ABSTRACT

The present invention provides a method of generating hydrogen by hydrolyzing a complex metal hydride in the presence of water and a catalyst, wherein the catalyst includes a noble metal and one of metal oxides, metalloid oxides and carbonaceous materials.

### BACKGROUND OF THE INVENTION

1. Field of the Invention

The present invention relates to a hydrogen generating method and a hydrogen generating apparatus; and, in particular, to a hydrogen generating method for hydrolyzing a complex metal hydride in the presence of water and a catalyst so as to generate hydrogen, and a hydrogen generating apparatus therefor.

2. Related Background Art

In modern society, hydrogen is an important chemical material which is utilized in a large amount in synthetic chemical industries, petroleum refining, and the like. On the other hand, technologies for utilizing hydrogen as clean energy are considered to assume an important position in order to overcome problems of energy and environment in future. Hence, fuel cells which store hydrogen and operate by using it as fuel have been under development.

Such a fuel cell is a battery which is actuated with a gas. At this time, energy obtained upon a reaction of hydrogen and oxygen is directly converted into electric energy. Since such a fuel cell has an efficiency much higher than that of conventional combustion engines, a car having a fuel cell is known as a ZEV (Zero Emission Vehicle).

Proposed as a hydrogen storing method, on the other hand, are a method in which hydrogen is compressed so as to be stored in a cylinder, a method in which hydrogen is cooled so as to become liquid hydrogen, a method in which hydrogen is adsorbed by active carbon, and a method utilizing a hydrogen-occluding alloy. Among these methods, the hydrogen-occluding alloy is considered to play an important role in a moving medium such as a fuel cell car. For the hydrogen-occluding alloy, however, there are also many problems to overcome, such as its heaviness (a small amount of occlusion per unit weight) due to its nature as an alloy, deterioration (the alloy turning into finer particles or changing its structure) upon repeated occlusions and releases, and securing of its resources when it includes rare metals.

Hence, attention has recently been given to a method proposed by Powerball Technologies, in which a halite type alkali hydride (sodium hydride) is hydrolyzed so as to generate hydrogen. When in contact with water, sodium hydride vigorously reacts therewith, so as to generate hydrogen. Therefore, sodium hydride is coated with a resin film, and the film is cut, so as to generate hydrogen. However, this method has had a problem that the maximum amount of hydrogen that can be generated from sodium hydride is 8.8 wt % (per 1 g of sodium hydride), whereby its energy density is not always sufficient as fuel for fuel cell cars. Also, there has been a problem in terms of safety since the halite type alkali hydride vigorously reacts with water when in contact therewith.

In view of such circumstances, sodium borohydride, which is a water-soluble complex metal hydride, has come to attention as a new hydrogen generating source. Hydrogen is generated from sodium borohydride according to the following hydrolysis reaction:

NaBH₄+2H₂O→NaBO₂+4H₂

and the like. Here, the maximum amount of hydrogen that can be generated from sodium borohydride is 21.3 wt % (per 1 g of sodium borohydride), so that the amount of hydrogen generation is at least twice as much as that in the above-mentioned method using sodium hydride, thereby satisfying the energy density required for fuel cell cars. Such hydrolysis of sodium borohydride has been known to accelerate in the presence of a catalyst. Conventionally known as such a catalyst are metal halides (NiCl₂, CoCl₂, and the like), colloidal platinum, active carbon, Raney nickel, and the like (“Sodium Borohydride, Its Hydrolysis and its Use as a Reducing Agent and in the Generation of Hydrogen,” H. I. Schlesinger et al., J. Am. Chem. Soc., vol. 75, p. 215-219 (1953)).

Even in the case using such a conventionally known catalyst, however, the hydrogen generating rate and amount have not been sufficient yet. Also, the case where the catalyst is water-soluble, as with a metal halide, has had problems that it is difficult for the catalyst to be utilized repeatedly, and that the hydrogen generating amount is hard to control.

### SUMMARY OF THE INVENTION

In view of the above-mentioned problems of background art, it is an object of the present invention to provide a hydrogen generating method which can achieve a sufficient hydrogen generating rate and amount when hydrolyzing a water-soluble complex metal hydride so as to generate hydrogen and makes it easier to repeatedly utilize the catalyst and control the hydrogen generating amount, and a hydrogen generating apparatus therefor.

The inventors have repeated diligent studies in order to achieve the above-mentioned object and, as a result, have found that, in a hydrolysis reaction for reacting a complex metal hydride and water so as to generate hydrogen, when a catalyst comprising a noble metal and at least one substance selected from the group consisting of metal oxides, metalloid oxides and carbonaceous materials is used, then the hydrogen generating rate and amount can sufficiently be improved, and it also becomes easier to utilize the catalyst repeatedly and control the hydrogen generating amount, thereby accomplishing the present invention.

Namely, the present invention provides a hydrogen generating method for generating hydrogen comprising a step of hydrolyzing a complex metal hydride in the presence of water and a catalyst, wherein the catalyst comprises a noble metal and at least one substance selected from the group consisting of metal oxides, metalloid oxides, and carbonaceous materials.

Also, the present invention provides a hydrogen generating apparatus comprising a first container in which a complex metal hydride and water are disposed, a second container in which a catalyst is disposed, and a pipe communicating the first and second containers to each other, the apparatus generating hydrogen by hydrolyzing the complex metal hydride in the presence of water and the catalyst, wherein the catalyst comprises a noble metal and at least one substance selected from the group consisting of metal oxides, metalloid oxides, and carbonaceous materials.

In the hydrogen generating method and apparatus of the present invention, the hydrolysis reaction of complex metal hydride is remarkably accelerated by a catalyst comprising a noble metal and at least one substance selected from the group consisting of metal oxides, metalloid oxides, and carbonaceous materials, so as to achieve a sufficient hydrogen generating rate and amount. Further, since the catalyst in accordance with the present invention is a water-insoluble solid, it can easily be isolated and collected so as to be utilized repeatedly, and the amount of catalyst contributing to the reaction can easily be increased and decreased so as to control the hydrogen generating amount.

Though the reason why the hydrolysis reaction of complex metal hydride is remarkably accelerated by the catalyst in accordance with the present invention is not clear, the inventors consider that it is achieved by a synergistic effect between the catalytic activity of the noble metal having a high oxidizing power and the catalytic activity of the metal oxide, metalloid oxide or carbonaceous material having many acid points.

Preferably, in the hydrogen generating method of the present invention, both of the substance and noble metal in the catalyst exist such as to be able to come into contact with the complex metal hydride and water in the hydrolyzing step. Further, the hydrogen generating apparatus of the present invention preferably further comprises a means for supplying the complex metal hydride and water into the second container simultaneously or successively whereby both of the substance and noble metal in the catalyst come into contact with the complex metal hydride and water.

Preferably, the complex metal halide in accordance with the present invention is at least one member selected from the group consisting of NaBH₄, NaAlH₄, LiBH₄, LiAlH₄, KBH₄, KAlH₄, Mg(BH₄)₂, Ca(BH₄)₂, Ba(BH₄)₂, Sr(BH₄)₂, and Fe(BH₄)₂. This is because of the fact that such a complex metal hydride has a high content of hydrogen, and reacts with water in the presence of the catalyst in accordance with the present invention, thereby efficiently generating hydrogen.

In the present invention, the metal oxide is preferably at least one metal oxide selected from the group consisting of titanium oxide, nickel oxide, cerium oxide, zeolite, alumina, zirconia, and manganese oxide;

the metalloid oxide is preferably a silicon oxide;

the carbonaceous material is preferably at least one carbonaceous material selected from the group consisting of active carbon, graphite, active char, coke, hard carbon, and soft carbon; and

the noble metal is preferably a platinum group element. Further, the substance is preferably a particle having an average particle size of 1000 μm or less and the noble metal is preferably a fine particle having an average particle size of 100 nm or less. With such a combination of a noble metal and a metal oxide, metalloid oxide, or carbonaceous material, the hydrolysis of complex metal hydride tends to progress more efficiently, so as to improve the hydrogen generating rate and amount more.

Preferably, in the hydrogen generating method and apparatus of the present invention, the catalyst comprises a lithium-containing combined metal oxide and a noble metal. With such a catalyst comprising a lithium-containing combined metal oxide and a noble metal, the hydrolysis reaction of complex metal hydride tends to accelerate more remarkably, thereby achieving a sufficient hydrogen generating rate and amount. The inventors consider that, due to such a catalyst, the above-mentioned hydrolysis reaction is remarkably accelerated by a synergistic effect between the noble metal having a high oxidizing power and the lithium-containing combined metal oxide that generates a surface activity upon insertion and desorption of lithium ion.

The lithium-containing combined metal oxide is preferably a particle having an average particle size of 1000 μm or less and comprising at least one lithium-containing combined metal oxide selected from the group consisting of lithium cobaltate, lithium niccolate, lithium manganate, lithium vanadate, and lithium chromate. Further, the noble metal in accordance with the present invention is preferably a fine particle having an average particle size of 100 nm or less and comprising a platinum group element. With such a combination of a noble metal and a lithium-containing combined metal oxide, the hydrolysis of complex metal hydride tends to progress more efficiently, so as to improve the hydrogen generating rate and amount more.

Preferably, in the hydrogen generating method and apparatus of the present invention, the catalyst is one in which the substance is caused to carry the noble metal by use of a highly-heated and highly-pressurized fluid maintained under a pressure of 1.013×10⁶ Pa (10 atm) or higher and at a temperature not lower than the boiling point of the fluid under this pressure. The inventors consider that, since the highly-heated and highly-pressurized fluid is used, the metal is carried as fine particles in such a catalyst, whereby its catalytic activity is enhanced.

Further, the highly-heated and highly-pressurized fluid is preferably a supercritical fluid. When a supercritical fluid is used as such, then the noble metal is carried as being uniformly dispersed with a superfine particle size of 10 nm or less in terms of average particle size, whereby the catalytic activity tends to improve further.

Preferably, in the hydrogen generating method and apparatus of the present invention, the catalyst is one subjected to reduction processing after the substance is caused to carry the noble metal. The inventors consider that the catalytic activity of such a catalyst is specifically enhanced since its surface is sufficiently metallized (turned into a metal element) by the reduction processing so as to increase its oxidizing power.

Preferably, the catalyst is one subjected to reduction processing in a reducing gas atmosphere at a temperature of 200 to 800° C. after the substance is caused to carry a fine particle of the noble metal having an average particle size of 5 nm or less. When the catalyst is subjected to reduction processing under such a condition, then it is more securely metallized so as to enhance its oxidizing power, and the catalytic activity tends to further improve because of the fact that the noble metal has an average particle size of 5 nm or less and thus is superfine as well.

The present invention will be more fully understood from the detailed description given hereinbelow and the accompanying drawings, which are given byway of illustration only and are not to be considered as limiting the present invention.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will be apparent to those skilled in the art from this detailed description.

### BRIEF DESCRIPTION OF THE DRAWINGS

FIG. 1 is a schematic view showing a preferred embodiment of the hydrogen generating apparatus in accordance with the present invention.

### DESCRIPTION OF THE PREFERRED EMBODIMENTS

In the following, preferred embodiments of the present invention will be explained in detail.

The hydrogen generating method of the present invention is a method for generating hydrogen by hydrolyzing a complex metal hydride in the presence of water and a catalyst, wherein the catalyst comprises a noble metal and at least one substance selected from the group consisting of metal oxides, metalloid oxides, and carbonaceous materials.

Namely, the catalyst in accordance with the present invention is one in which a noble metal and at least one substance selected from the group consisting of metal oxides, metalloid oxides, and carbonaceous materials coexist. The mode of coexistence may be one in which the substance is used as a carrier and is caused to carry the noble metal, one in which they are mixed, and the like. Among them, one in which a carrier made of the substance is caused to carry the noble metal is preferable since the catalytic activity tends to become higher. Preferably, the substance is in a particle form, whereas the noble metal is in a fine particle form, since the catalytic activity tends to become further higher in this case.

Examples of such metal oxides include oxides of noble metal elements (Pt, Pd, Rh, Ru, Au, and the like) and base metal elements (Y, La, Ce, Pr, Nd, Eu, Gd, Tb, Dy, Ho, Er, Tm, Yb, Lu, Ca, Mg, Al, K, Ti, Cr, Mn, Fe, Co, Ni, Cu, Ga, Rb, Sr, Zr, Nb, Mo, In, Sn, Cs, Ba, Ta, W, and the like). Among them, a single oxide or combined oxide of at least one metal selected from the group consisting of Ti, Al, Ce, Zr, Fe, Mn, Ni, Zn, Cu, Mg, and Co is preferable. Titanium oxide (titania), alumina, silica-alumina, cerium oxide (ceria), zirconia, titania-zirconia, ceria-zirconia, zeolite, iron oxide, manganese oxide, nickel oxide, zinc oxide, and copper oxide are more preferable. In particular, titaniumoxide, nickeloxide, ceriumoxide, zeolite, alumina, zirconia, and manganese oxide are preferable. The metal oxide in accordance with the present invention may contain a plurality of metal elements as in zeolite, titania-zirconia, and ceria-zirconia, and may further contain nonmetal elements.

When such a metal oxide is used, then this substance itself also acts as a catalyst, so that the hydrolysis of complex metal hydride is remarkably accelerated due to a synergistic effect with the noble metal mentioned later in particular, whereby a sufficient hydrogen generating rate and amount is achieved. Though the reason why a metal oxide acts as a catalyst is unclear, the inventors consider that, since many acid points exist in the metalloid oxide as well, the pH of a reaction system in which the complex metal hydride and water react with each other so as to generate hydrogen while yielding an alkaline reaction product is lowered, whereby a catalytic activity is generated.

More preferably, the metal oxide in accordance with the present invention is a lithium-containing combined metal oxide. An example of such a lithium-containing combined metal oxide is a combined metal oxide (combined oxide) in which lithium oxide and at least one metal oxide excluding the same and/or metalloid oxide form a compound. In particular, lithium cobaltate (LiCoO₂), lithium niccolate (LiNiO₂), lithium manganese (LiMnO₂, LiMn₂O₄), lithium vanadate (LiVO₂, LiV₂O₄), and lithium chromate (LiCrO₂) are preferable. Such a lithium-containing combined metal oxide may be a combined metal oxide comprising a lithium oxide and at least two kinds of metal oxides excluding the same. For example, those in which a part of cobalt in lithium cobaltate is substituted by other elements (e.g., Ni, Mn, Al, Fe, and B), those in which a part of nickel in lithium niccolate is substituted by other elements (e.g., Co, Mn, Al, Fe, and B), and those in which a part of manganese in lithium manganese is substituted by other elements (e.g., Ni, Co, Al, Fe, and B) are also suitably usable. Though the reason why a lithium-containing combined metal oxide acts as a catalyst is unclear, the inventors consider that an electrically active state is generated in the process of repeatedly inserting and desorbing lithium ion in the lithium-containing combined metal oxide, so as to supply electrons necessary for generating hydrogen in the reaction of the complex metal hydride and water on the noble metal surface, whereby a very high catalytic reaction activity is exhibited.

The metal oxide in accordance with the present invention is a particle preferably having an average particle size of 1000 μm or less, more preferably 100 μm to 10 nm, particularly preferably 10 μm to 10 nm. If the average particle size exceeds 1000 μm, then the surface area of particle tends to lower, whereby a sufficient catalytic activity may not be obtained. Preferably, the specific surface area of metal oxide is about 1 to 1000 m²/g. When the average particle size is relatively large, the metal oxide is preferably a porous particle.

Examples of the metalloid oxides include oxides of metalloid elements (Si, Ge, As, Sb, and the like), among which silicon oxide (silica gel) is preferable. If such a metalloid oxide is used, then this substance itself also acts as a catalyst, so that the hydrolysis of complex metal hydride is remarkably accelerated due to a synergistic effect with the noble metal mentioned later in particular, whereby a sufficient hydrogen generating rate and amount is achieved. Though the reason why a metalloid oxide acts as a catalyst is also unclear, the inventors consider that, since many acid points exist in the metalloid oxide as well, the pH of a reaction system in which the complex metal hydride and water react with each other so as to generate hydrogen while yielding an alkaline reaction product is lowered, whereby a catalytic activity is generated.

The metalloid oxide in accordance with the present invention is a particle preferably having an average particle size of 1000 μm or less, more preferably 100 μm to 10 nm, particularly preferably 10 μm to 10 nm. If the average particle size exceeds 1000 μm, then the surface area of particle tends to lower, whereby a sufficient catalytic activity may not be obtained. Preferably, the specific surface area of metalloid oxide is about 0.1 to 500 m²/g. When the average particle size is relatively large, the metal oxide is preferably a porous particle.

Preferred as the carbonaceous material are active carbon, graphite, active char, coke, hard carbon (carbon which is hard to become graphite), and soft carbon (carbon which easily becomes graphite). When such a carbonaceous material is used, this substance itself also acts as a catalyst, so that the hydrolysis of complex metal hydride is remarkably accelerated due to a synergistic effect with the noble metal mentioned later in particular, whereby a sufficient hydrogen generating rate and amount is achieved. Though the reason why a carbonaceous material acts as a catalyst is unclear, the inventors consider that, since many acid points exist in the carbonaceous material as well, the pH of a reaction system in which the complex metal hydride and water react with each other so as to generate hydrogen while yielding an alkaline reaction product is lowered, whereby a catalytic activity is generated.

The carbonaceous material in accordance with the present invention is a particle preferably having an average particle size of 1000 μm or less, more preferably 100 μm to 10 nm, particularly preferably 10 μm to 10 nm. If the average particle size exceeds 1000 μm, then the surface area of particle tends to lower, whereby a sufficient catalytic activity may not be obtained. Preferably, the specific surface area of metal oxide is about 1 to 4000 m²/g. The carbonaceous material is preferably a porous particle.

The form of the substance in accordance with the present invention is not restricted in particular, and forms such as powder, pellet, monolith, sheet, and fiber may be selected according to the condition of use.

In the catalyst in accordance with the present invention, the above-mentioned substance and a noble metal coexist. Examples of such a noble metal include Pt, Pd, Rh, Ru, Ir, Os, Au, and Ag, among which platinum group elements (Pt, Pd, Rh, Ru, Ir, and Os) are preferable. When such a noble metal is used together with the above-mentioned substance, then the hydrolysis of complex metal hydride is remarkably accelerated, whereby a sufficient hydrogen generating rate and amount is achieved. Though the reason why a noble metal acts as a catalyst is unclear, the inventors consider that, since the noble metal has a high oxidizing power, a catalytic activity occurs in a reaction system in which the complex metal hydride and water react with each other so as to generate hydrogen.

As the noble metal in accordance with the present invention, one having an average particle size smaller than that of a particle made of the above-mentioned substance is desirable. It is a fine particle preferably having an average particle size of 100 nm or less, more preferably 10 nm or less, further preferably 5 nm or less, particularly preferably2 nm or less. If the average particle size exceeds 100 nm, then the surface area of particle tends to decrease so that a sufficient catalytic activity may not be obtained. Though the noble metal may partly contain a noble metal compound such as a noble metal oxide, it is preferably a noble metal element since a higher oxidizing power is obtained thereby.

The content of noble metal in the catalyst in accordance with the present invention is preferably 0.01% to 20% by weight, more preferably 0.05% to 5% by weight, particularly preferably 0.5% to 5% by weight, based on the total weight of catalyst. If the noble metal content is less than 0.01% by weight, then there is a tendency that a sufficient catalytic action may not be obtained by the noble metal, whereby a sufficient hydrogen yield may not be achieved.

The method of causing the noble metal to coexist with the above-mentioned substance is not restricted in particular. For example, the noble metal and/or noble metal precursor (halide, nitrate, carbonate, acetylacetonate, tetraammine salt, alkoxide, or the like of the noble metal) can be used so as to cause a carrier made of the above-mentioned substance to carry the noble metal by means of a technique such as so-called immersion, sedimentation, kneading, or ion-exchange, thereby yielding the catalyst in accordance with the present invention. Preferably, so-called highly-heated and highly-pressurized method explained in the following is used, and the use of so-called supercritical method is particularly preferable.

The highly-heated and highly-pressurized method is a method in which the above-mentioned substance is caused to carry the noble metal by use of a highly-heated and highly-pressurized fluid maintained under a pressure of 1.013×10⁶ Pa (10 atm) or higher, more preferably 1.520×10⁶ Pa (15 atm) or higher, and at a temperature not lower than the boiling point under this pressure. More specifically, it is:

(i) a method in which a solution containing a noble metal and/or noble metal precursor and a solvent are brought into contact with a carrier made of the above-mentioned substance in a state where the solvent becomes the above-mentioned highly-heated and highly-pressurized fluid, so as to cause the carrier surface to carry a fine particle of the noble metal and/or noble metal precursor; or

(ii) a method in which a carrier made of the above-mentioned substance is caused to temporarily carry a noble metal and/or noble metal precursor by means of a technique such as so-called immersion, sedimentation, kneading, or ion-exchange using the noble metal and/or noble metal precursor, they are dried if necessary, and then a solvent is brought into contact with the carrier in a state where it becomes the above-mentioned highly-heated and highly-pressurized fluid, whereby the carrier surface is caused to carry a fine particle of the noble metal and/or noble metal precursor.

Since such a highly-heated and highly-pressurized fluid tends to have a dissolving capacity similar to that of a liquid and a diffusivity and viscosity similar to that of a gas, it can distribute the noble metal rapidly and uniformly with a very fine state into deep parts of holes of the carrier and holes having a very fine diameter. The dissolving capacity can be adjusted by temperature, pressure, entrainer (additive), and the like. If such a highly-heated and highly-pressurized fluid is used, then the noble metal is carried with a fine particle size of 10 nm or less (preferably 5 nm or less, more preferably 2 nm or less) while being evenly dispersed under a uniform pressure, so that the catalytic activity improves further, whereby the hydrogen generating rate and amount tend to improve further.

The supercritical method is a method in which a supercritical fluid disclosed in the inventors, International Publication No. WO99/10167 is used for causing the above-mentioned substance to carry the noble metal. More specifically, it is:

(i) a method in which a solution containing a noble metal and/or noble metal precursor and a solvent are brought into contact with a carrier made of the above-mentioned substance in a state where the solvent becomes a supercritical fluid, so as to cause the carrier surface to carry a fine particle of the noble metal and/or noble metal precursor; or

(ii) a method in which a carrier made of the above-mentioned substance is caused to temporarily carry a noble metal and/or noble metal precursor by means of a technique such as so-called immersion, sedimentation, kneading, or ion-exchange using the noble metal and/or noble metal precursor, they are dried if necessary, and then a solvent is brought into contact with the carrier in a state where it becomes a supercritical fluid, whereby the carrier surface is caused to carry a fine particle of the noble metal and/or noble metal precursor.

Here, the supercritical fluid refers to a fluid heated to its critical temperature or higher. Therefore, the state where a solvent becomes a supercritical fluid refers to a state where a solvent is heated to the critical temperature of solvent or higher. Though the pressure is not restricted in particular, it is preferably a critical pressure or higher. Since such a supercritical fluid has a dissolving capacity similar to that of a liquid and a diffusivity and viscosity similar to that of a gas, it can distribute the noble metal rapidly and uniformly with a very fine state into deep parts of holes of the carrier and holes having a very fine diameter. The dissolving capacity can be adjusted by temperature, pressure, entrainer (additive), and the like. If such a supercritical fluid is used, then the noble metal is carried with a fine particle size of 10 nm or less (preferably 5 nm or less, more preferably 2 nm or less) while being evenly dispersed under a uniform pressure, so that the catalytic activity improves further, whereby the hydrogen generating rate and amount tend to improve further.

The solvent that becomes such a highly-heated and highly-pressurized fluid or supercritical fluid is not restricted in particular. Its examples include hydrocarbons such as methane, ethane, propane, butane, ethylene, and propylene; monools such as methanol, ethanol, and isopropanol; glycols such as ethylene glycol and propylene glycol; ketones such as acetone and acetylacetone; ethers such as dimethyl ether; carbon dioxide; water; ammonia; chlorine; chloroform; and Freons. Also, alcohols such as methanol, ethanol, and propanol; ketones such as acetone, ethylmethylketone, and acetylacetone; aromatic hydrocarbons such as benzene, toluene, and xylene; and the like may be used as an entrainer for increasing the solubility of fluid into the noble metal and/or noble metal precursor.

After the carrier is caused to carry the noble metal and/or noble metal precursor as mentioned above, firing processing may be carried out if necessary. Though the condition for such firing processing is not restricted in particular, a condition such as heating at a temperature of 200 to 800° C. (preferably 350 to 800° C.) for 1 to 10 hours in the atmosphere of air, nitrogen, or the like is employed, for example.

Preferably, after the carrier made of the above-mentioned substance is caused to carry a fine particle of noble metal and/or noble metal precursor, thus obtained carrier carrying the fine particle of noble metal is subjected to reduction processing in the present invention. The method of such reduction processing is not restricted in particular. A preferably employable example of this method is:

(i) a method in which the carrier carrying the fine particle of noble metal is heated in a reducing gas atmosphere; or

(ii) a method in which the carrier carrying the fine particle of noble metal is brought into contact with a reducing chemical. When the carrier carrying the fine particle of noble metal is subjected to reduction processing as such, its surface is sufficiently metallized (turned into a metal element), so as to increase its oxidizing power, whereby its catalytic activity tends to increase specifically.

Preferable reduction processing will now be explained in further detail. In the reduction processing method (i), preferred examples of the reducing gas are gases containing reducing components such as hydrogen, carbon monoxide, hydrocarbons (such as methane), and aldehydes (such as acetaldehyde and formaldehyde), among which hydrogen-containing gases are preferable in particular. The content of reducing component in such a reducing gas is preferably 0.1% by volume or greater, more preferably 1% by volume or greater. In the case where the reducing component is hydrogen, the hydrogen content is preferably 1% to 20% by volume, more preferably 2% to 10% by volume. If the content of reducing component (hydrogen or the like) is less than the lower limit mentioned above, then the reduction processing tends to become insufficient, whereby its catalytic activity may not improve sufficiently. If the hydrogen content exceeds the upper limit mentioned above, by contrast, then its handling tends to become difficult in terms of safety. As a gas other than the reducing component in the reducing gas, nitrogen and inert gases are preferred.

In the reduction processing method (i), the carrier carrying the fine particle of noble metal is heated in the reducing gas atmosphere preferably at a temperature of 200 to 800° C. (more preferably 300 to 600° C.) for preferably 1 to 10 hours, so as to perform reduction processing. If this temperature is lower than the lower limit mentioned above, then the reduction processing tends to become insufficient, whereby the catalytic activity may not improve sufficiently. If the temperature exceeds the upper limit mentioned above, by contrast, then a heat history may apply to the carrier, or sintering may occur in the carrier metal, whereby there is a possibility of lowering the activity of catalyst.

In the reduction processing method (ii), preferred examples of the reducing chemical are solutions containing reducing compounds such as hydrazine, ethylene glycol, hydrogen-containing inorganic compounds (chemical hydrides such as sodium borohydride). The content of reducing compound in such a reducing chemical is preferably 1% by weight or greater. As a component (solvent) other than the reducing compound in the reducing chemical, water is preferable.

In the reduction processing method (ii), the carrier carrying the fine particle of noble metal is brought into contact with (e.g., immersed in) the reducing chemical preferably for 10 minutes to 12 hours, so as to carry out reduction processing, and drying processing and/or firing processing may be carried out if necessary. The condition for such firing processing is not restricted in particular. For example, a condition such as heating at a temperature of 150 to 400° C. in an atmosphere of air, nitrogen, or the like may be employed.

In the hydrogen generating method of the present invention, a complex metal hydride and water are brought into contact with each other in the presence of a catalyst comprising a noble metal and at least one substance selected from the group consisting of metal oxides, metalloid oxides, and carbonaceous materials as mentioned above. As a consequence, the hydrolysis reaction of complex metal hydride is remarkably accelerated, whereby hydrogen is generated at a high yield with a sufficient hydrogen generating rate and amount.

As such a complex metal hydride, NaBH₄, NaAlH₄, LiBH₄, LiAlH₄, KBH₄, KAlH₄, Mg(BH₄)₂, Ca(BH₄)₂, Ba(BH₄)₂, Sr(BH₄)₂, and Fe(BH₄)₂ are preferable since they have a high hydrogen content, so that hydrogen is efficiently generated therefrom in the presence of the catalyst. These complex metal hydrides may be used as a single species or in a combination of a plurality of species.

More preferably, the complex metal hydride is NaBH₄ since it is obtained at a low cost, its reactivity with water is low by itself, and the theoretical volume of its hydrogen generation is 21.3 wt %, thus being high.

In the hydrogen generating method of the present invention, water is used together with the complex metal hydride, which is a raw material. It will be sufficient if the amount of water is not lower than the stoichiometric amount with respect to the complex metal hydride, which is a raw material. The amount of water is preferably 0.1 to 100 mol, more preferably 1.5 to 100 mol, per 1 mol of complex metal hydride. If the amount of water is lower than the lower limit mentioned above, then there is a tendency that a higher hydrogen generating amount may not be obtained. If the amount of water is greater than 100 mol/mol, then there is a tendency that the effect of addition may not improve well, thus becoming uneconomical.

The reaction system in the hydrogen generating method of the present invention may contain components other than the complex metal hydride, water, and catalyst. Examples of the other components include gases (nitrogen, CO₂, Ar, and the like) which are inert to the reaction. On the other hand, it is preferred that oxygen be excluded as much as possible since generated hydrogen tends to be burned easily if oxygen exists.

For preventing an initial reaction of the complex metal hydride and water from occurring, it is preferred that an alkali (sodium hydroxide or the like) be added to the solution of complex metal hydride and water by about 10⁻⁴ mol to 0.1 mol per 1 liter of aqueous solution.

In the hydrogen generating method of the present invention, the complex metal hydride may be hydrolyzed in the presence of a solution comprising an acid and water, so as to generate hydrogen. It tends to accelerate the hydrolysis reaction of complex metal hydride, whereby hydrogen may be generated at a higher yield with a sufficient hydrogen generating amount. Preferably employable as such an acid are organic acids such as acetic acid, oxalic acid, carboxylic acid, and lactic acid and inorganic acids such as hydrochloric acid, sulfuric acid, nitric acid, sulfurous acid, hydrogen sulfide, and phosphoric acid, among which organic acids are more preferable from the viewpoint of safety. Such acids may be used as a single species or in a combination of a plurality of species. The acid content in the solution comprising the acid and water is preferably 2% to 98% by weight, more preferably 10% to 95% by weight. If the acid content is less than the lower limit mentioned above, then there is a tendency that the effect of addition of acid may not be obtained sufficiently. If the acid content exceeds the upper limit mentioned above, by contrast, then the amount of water contributing to hydrolysis may be lower, whereby there is a tendency that a higher hydrogen generating amount may not be obtained. If the employed acid has a solubility to water lower than the above-mentioned upper limit, then it is preferred that the acid content in the solution comprising the acid and water be lower than this solubility.

Though the reaction condition in the hydrogen generating method of the present invention is not limited in particular, temperature is preferably 0 to 200° C., more preferably 0 to 100° C., particularly preferably 10 to 80° C. If the reaction temperature is lower than 0° C., then water may freeze, whereby the hydrogen generating rate tends to lower. If the temperature is higher than 200° C., by contrast, then water is likely to become vapor even under a pressurizing condition, whereby the hydrogen generating rate tends to lower.

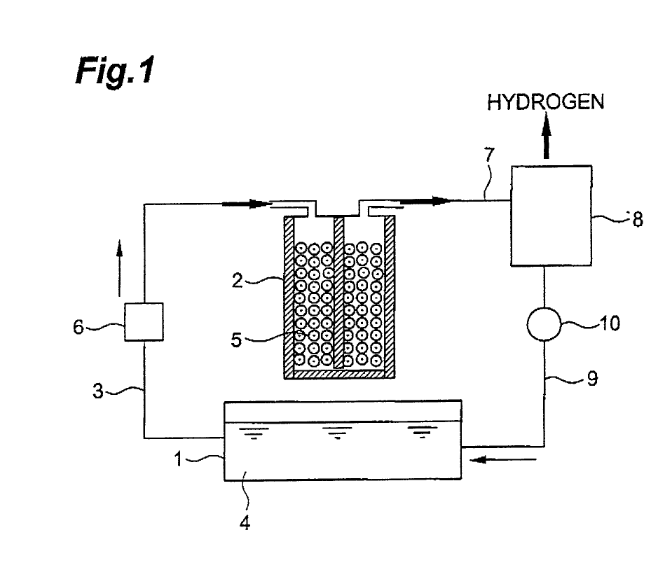

A preferred embodiment of the hydrogen generating apparatus in accordance with the present invention will now be explained. FIG. 1 is a schematic view showing an example of preferred embodiment of the hydrogen generating apparatus in accordance with the present invention. This apparatus comprises a storage tank (first container) 1, a catalyst container (second container) 2, and a pipe 3 communicating them to each other. An aqueous solution 4 in which a complex metal hydride and water are mixed is held in the storage tank 1, whereas a hydrogen generating catalyst 5 in accordance with the present invention is held in the catalyst container 2.

In the apparatus shown in FIG. 1, the pipe 3 is provided with a throttle 6 for regulating the amount of supply of complex metal hydride aqueous solution 4 to the catalyst container 2, whereas a hydrogen separator 8 for separating the unreacted complex metal hydride and generated hydrogen from each other by way of a pipe 7 is installed in the catalyst container 2. Also, a pipe 9 for returning the complex metal hydride isolated by the hydrogen separator 8 to the storage tank 1 is provided, whereas a compressor 10 for stably supplying the complex metal hydride aqueous solution 4 is connected to the pipe 9.

According to such a hydrogen generating apparatus, the complex metal hydride aqueous solution 4 is supplied from the storage tank 1 to the catalyst container 2 with its amount of supply being adjusted by the throttle 6 and compressor 10. As the complex metal hydride and water come into contact with each other in the presence of catalyst 5, hydrogen is generated at a high yield. Hydrogen obtained in this apparatus is isolated by the hydrogen separator 8, and then is supplied to a reaction cell (not depicted) for a fuel cell, for example. Therefore, if a predetermined amount of the complex metal hydride aqueous solution 4 is supplied to the catalyst container 2 according to an amount of energy to be taken out as electric power, then the amount of hydrogen supplied to the reaction cell for a fuel cell can be adjusted, whereby a required electric output can be obtained.

Since the catalyst 5 within the catalyst container 2 is a water-insoluble solid in the apparatus of the present invention, it can easily be isolated and collected so as to be utilized repeatedly, and the amount of catalyst contributing to the reaction can easily be increased or decreased so as to control the hydrogen generating amount. If the unreacted complex metal hydride isolated by the hydrogen separator 8 is returned to the storage tank 1 by way of the pipe 9, then the complex metal hydride can further be utilized effectively.

While a preferred embodiment of the hydrogen generating apparatus in accordance with the present invention is explained in the foregoing, the apparatus of the present invention should not be restricted to the above-mentioned embodiment. For example, the complex metal hydride and water may be prepared separately, so as to be supplied to the catalyst container simultaneously or successively. Also, the catalyst may be added into the complex metal hydride aqueous solution while in a removable manner, so as to adjust the hydrogen generating amount according to the amount of catalyst.

### EXAMPLES

In the following, the present invention will be explained more specifically with reference to Examples and Comparative Examples, though the present invention is not restricted by the following Examples.

### Examples 1 to 4

Into 5 ml of acetone, 500 mg of platinum acetylacetonate were dissolved. The resulting solution was introduced into an autoclave, into which 1 g of titania powder (manufactured by Sachtleben Chemie GmbH, UV100) and 30 g of dry ice were further added. After being tightly closed, the autoclave was heated and pressurized at a temperature of 150° C. under a pressure of 300 kg/cm², and held for 2 hours, so as to cause the titania powder to carry platinum acetylacetonate while in a state where carbon dioxide is a supercritical fluid. Then, the titania powder was held at 105° C. for 1 hour, so as to yield a catalyst in which platinum is carried on titania (with a platinum content of 1.3 wt %).

Using thus obtained catalyst, the hydrogen generating rate and amount were determined as follows. Namely, after each amount of catalyst shown in Table 1 and 50 mg of sodium borohydride were packed into an Erlenmeyer flask having a volume of 100 ml, 5 ml of water were added dropwise thereto at room temperature (about 20° C.) by use of a syringe, and the hydrogen generating rate and amount were determined from the change in level of the volumetric burette in a gas analyzer (product code: 6071-4) made by Sibata Scientific Technology, Ltd. Here, the amount of hydrogen generated during the time shown in Table 1 (120 minutes at the maximum) from the starting of the test was measured, and was defined as a measured value of hydrogen generating amount. The hydrogen generating rate (the amount of hydrogen generated per 1 g of NaBH₄ per second) was calculated from the hydrogen generating amount at the lapse of 1 minute after starting the test.

The respective average particle sizes of the carrier particle and the carried fine particle of noble metal were determined by TEM observation, SEM observation, or X-ray diffraction. When determining the particle size by X-ray diffraction, X-ray diffraction apparatus RAD-B manufactured by Rigaku Corporation was used according to the following process.

Namely, the catalyst was packed into a sample cell made of glass, CuKα turned monochromatic by a graphite monochrometer was used as a ray source, and the wide-angle X-ray diffraction intensity curve was measured according to reflection type diffractometer method. Then, the particle size (thickness of crystal in a direction perpendicular to the lattice plane) L_{c} was determined by the following Scherrer's expression:

L_{c}=Kλ/β cosθ (where K=0.90)

according to the half width β, wavelength λ, and Bragg angle θ of the diffraction ray caused by the lattice plane.

The hydrogen generating rate and amount obtained by the above-mentioned measurement are shown in Table 1 together with data concerning the catalyst employed.

### Examples 5 and 6

Into 33 ml of platinum P salt solution (a nitrate solution of platinum having a platinum content of 50 g/l, manufactured by Tanaka Kikinzoku Kogyo K.K.), 100 g of titania powder similar to that in Example 1 were immersed, so as to cause the titania powder to carry the nitrate of platinum. Then, the titania powder was held at 250° C. for 5 hours, so as to be dried. Thereafter, the dried powder was fired for 2 hours in air at 450° C., and subsequently was held for 3 hours in hydrogen at 300° C., so as to be reduced, whereby a catalyst in which platinum was carried on titania (having a platinum content of 1.64 wt %) was obtained. Then, using thus obtained catalyst, the hydrogen generating rate and amount were determined as in Example 1, which are shown in Table 1 together with data concerning the catalyst employed.

### Example 7

A catalyst in which palladium was carried on titania (having a palladium content of 1.3 wt %) was obtained as in Example 1 except that 500 mg of palladium acetylacetonate were used in place of platinum acetylacetonate. Then, using thus obtained catalyst, the hydrogen generating rate and amount were determined as in Example 1, which are shown in Table 1 together with data concerning the catalyst employed.

### Examples 8 and 9

A catalyst in which palladium or ruthenium was carried on titania (having a palladium content of 1.5 wt % in Example 8, a ruthenium content of 1.5 wt % in Example 9) was obtained as in Example 5 except that 30.5 ml of dinitrodiammine palladium (II) nitric acid solution (having a Pd content of 50 g/l, manufactured by Tanaka Kikinzoku Kogyo K.K.) (Example 8) or 30.5 ml of ruthenium nitrate solution (having an Ru content of 50 g/l, manufactured by Tanaka Kikinzoku Kogyo K.K.) (Example 9) were used in place of platinum P salt solution. Then, using thus obtained catalyst, the hydrogen generating rate and amount were determined as in Example 1, which are shown in Table 1 together with data concerning the catalyst employed.

### Examples 10 and 11

A catalyst in which platinum was carried on γ-alumina (having a platinum content of 1.3 wt %) was obtained as in Example 1 except that 1 g of γ-alumina powder (manufactured by Nikki-Universal Co., Ltd.) was used in place of titania powder. Then, using thus obtained catalyst, the hydrogen generating rate and amount were determined as in Example 1, which are shown in Table 1 together with data concerning the catalyst employed.

### Examples 12 and 13

A catalyst in which platinum was carried on γ-alumina (having a platinum content of 1.64 wt %) was obtained as in Example 5 except that 100 g of γ-alumina powder were used in place of titania powder. Then, using thus obtained catalyst, the hydrogen generating rate and amount were determined as in Example 1, which are shown in Table 1 together with data concerning the catalyst employed.

### Examples 14 and 15

A catalyst in which platinum was carried on ceria-zirconia solid solution or titania-zirconia solid solution (having a platinum content of 1.3 wt % in Example 14, 1.5 wt % in Example 15) was obtained as in Example 1 except that 1 g of ceria-zirconia solid solution powder (manufactured by the method described in Japanese Patent Application Laid-Open No. HEI 9-221304, in which ceria and zironia had a molar ratio of 1:1) or 1 g of titania-zirconia solid solution powder (manufactured by the following method described in Japanese Patent Application No. HEI 11-068347, in which titania and zironia had a weight ratio of 7:3) was used in place of titania powder. Then, using thus obtained catalyst, the hydrogen generating rate and amount were determined as in Example 1, which are shown in Table 1 together with data concerning the catalyst employed.

(Method of Making Titania-Zirconia Solid Solution Powder)

Into a mixture constituted by 305 g of 28% tetrachlorotitanium solution and 200 g of 18% zirconyl oxynitrate aqueous solution, 1000 g of ion-exchanged water were added, and they were further mixed. The resulting mixed liquid was neutralized with 1456 g of 8% aqueous ammonia solution being added thereto. Thus obtained gel was dried at 150° C., calcinated at 400° C., and further fired at 500° C., whereby titania-zirconia solid solution powder was obtained.

### Example 16

A catalyst in which platinum was carried on silicon oxide powder (having a platinum content of 1.5 wt %) was obtained in as in Example 5 except that 100 g of silicon oxide powder (manufactured by UOP) were used in place of titania powder. Then, using thus obtained catalyst, the hydrogen generating rate and amount were determined as in Example 1, which are shown in Table 1 together with data concerning the catalyst employed.

TABLE 1

| | | | | | AVERAGE | | | |
|---|---|---|---|---|---|---|---|---|
| | | | AMOUNT | | PARTICLE | | | HYDROGEN |
| | | | OF | | SIZE | AVERAGE | HYDROGEN | GENERATING |
| | CATALYST | AMOUNT | CARRIED | AMOUNT | OF CARRIED | PARTICLE | GENERATING | AMOUNT |
| | (CARRIER) + | OF | NOBLE | OF | NOBLE | SIZE | RATE | [wt %] |
| | (CARRIED NOBLE | CATALYST | METAL | OXIDE | METAL | OF OXIDE | [gg⁻¹ | (PER 1 g OF |
| | METAL) | [mg] | [mg] | [mg] | [nm] | [nm] | sec⁻¹] | NaBH₄) |
| EXAMPLE | TiO₂ + PLATINUM | 390 | 5 | 385 | 1 OR LESS | 50 | 1.93 × 10⁻² | 11.6 |
| 1 | | | | | | | | |
| EXAMPLE | TiO₂ + PLATINUM | 39 | 0.5 | 38.5 | 1 OR LESS | 50 | 8.78 × 10⁻⁴ | 12.8 |
| 2 | | | | | | | | |
| EXAMPLE | TiO₂ + PLATINUM | 3.9 | 0.05 | 3.85 | 1 OR LESS | 50 | 1.2 × 10⁻⁴ | 19.7 |
| 3 | | | | | | | | |
| EXAMPLE | TiO₂ + PLATINUM | 0.5 | 0.0065 | 0.494 | 1 OR LESS | 50 | 4.22 × 10⁻⁵ | 14.3 |
| 4 | | | | | | | | |
| EXAMPLE | TiO₂ + PLATINUM | 3.1 | 0.05 | 3.05 | 2.0 | 50 | 6.44 × 10⁻⁵ | 13.0 |
| 5 | | | | | | | | |
| EXAMPLE | TiO₂ + PLATINUM | 0.5 | 0.0082 | 0.492 | 2.0 | 50 | 2.72 × 10⁻⁵ | 7.58 |
| 6 | | | | | | | | |
| EXAMPLE | TiO₂ + PALLADIUM | 3.8 | 0.049 | 3.75 | 1 OR LESS | 50 | 4.59 × 10⁻⁵ | 7.93 |
| 7 | | | | | | | | |
| EXAMPLE | TiO₂ + PALLADIUM | 33 | 0.5 | 32.5 | 1.8 | 50 | 9.32 × 10⁻⁵ | 8.22 |
| 8 | | | | | | | | |
| EXAMPLE | TiO₂ + RUTHENIUM | 33 | 0.5 | 32.5 | 2.2 | 50 | 7.99 × 10⁻⁵ | 17.07 |
| 9 | | | | | | | | |
| EXAMPLE | γ-Al₂O₃ + PLATINUM | 3.9 | 0.05 | 3.85 | 1 OR LESS | 160 | 4.18 × 10⁻⁵ | 5.99 |
| 10 | | | | | | | | |
| EXAMPLE | γ-Al₂O₃ + PLATINUM | 0.5 | 0.0065 | 0.4935 | 1 OR LESS | 160 | 1.85 × 10⁻⁵ | 2.82 |
| 11 | | | | | | | | |
| EXAMPLE | γ-Al₂O₃ + PLATINUM | 305 | 5 | 300 | 2.0 | 160 | 1.07 × 10⁻³ | 17.9 |
| 12 | | | | | | | | |
| EXAMPLE | γ-Al₂O₃ + PLATINUM | 3.1 | 0.05 | 3.05 | 2.0 | 160 | 2.09 × 10⁻⁵ | 5.34 |
| 13 | | | | | | | | |
| EXAMPLE | CeO₂—ZrO₂ + | 33.3 | 0.5 | 32.8 | 1 OR LESS | 100 | 2.22 × 10⁻⁴ | 9.10 |
| 14 | PLATINUM | | | | | | | |
| EXAMPLE | TiO₂—ZrO₂ + PLATINUM | 25.2 | 0.378 | 24.8 | 1 OR LESS | 35 | 2.18 × 10⁻⁴ | 20(40 min) |
| 15 | | | | | | | | |
| EXAMPLE | SiO₂ + PLATINUM | 33.3 | 0.5 | 32.8 | 1.5 | 50000 | 9.51 × 10⁻⁵ | 13.0 |
| 16 | | | | | | | | |

### Examples 17 to 21

The hydrogen generating rate and amount were determined as in Example 1 except that the following catalysts were used:

platinum-active carbon (having a specific surface area of 719 m²/g) manufactured by Wako Pure Chemical Industries, Ltd. in Example 17;

palladium-active carbon (having a specific surface area of 769 m²/g) manufactured by Wako Pure Chemical Industries, Ltd. in Example 18; and

ruthenium-active carbon (having a specific surface area of 832 m²/g) manufactured by Wako Pure Chemical Industries, Ltd. in Examples 19 to 21.

Thus obtained data are shown in Table 2 together with data concerning the catalysts used.

TABLE 2

| | | | | AMOUNT | | AVERAGE | | |
|---|---|---|---|---|---|---|---|---|
| | | | | OF | AVERAGE | PARTICLE | | |
| | | | AMOUNT | CAR- | PARTICLE | SIZE OF | | HYDROGEN |
| | | | OF | BON- | SIZE | CAR- | HYDROGEN | GENERATING |
| | CATALYST | AMOUNT | CARRIED | ACEOUS | OF CARRIED | BON- | GENERATING | AMOUNT |
| | (CARRIER) + | OF | NOBLE | MATER- | NOBLE | ACEOUS | RATE | [wt %] |
| | (CARRIED NOBLE | CATALYST | METAL | IAL | METAL | MATERIAL | [gg⁻¹ | (PER 1 g OF |
| | METAL) | [mg] | [mg] | [mg] | [nm] | [nm] | sec⁻¹] | NaBH₄) |
| EXAMPLE | ACTIVE CARBON + | 1 | 0.05 | 0.95 | 3.53 | 20000 | 2.75 × 10⁻⁵ | 5.1 |
| 17 | PLATINUM | | | | | | | |
| EXAMPLE | ACTIVE CARBON + | 1 | 0.1 | 0.9 | 3.35 | 20000 | 3.68 × 10⁻⁵ | 4.82 |
| 18 | PALLADIUM | | | | | | | |
| EXAMPLE | ACTIVE CARBON + | 1 | 0.05 | 0.95 | 2.09 | 20000 | 5.95 × 10⁻⁵ | 7.57 |
| 19 | RUTHENIUM | | | | | | | |
| EXAMPLE | ACTIVE CARBON + | 2 | 0.1 | 1.9 | 2.09 | 20000 | 1.21 × 10⁻⁴ | 20 (1 HR) |
| 20 | RUTHENIUM | | | | | | | |
| EXAMPLE | ACTIVE CARBON + | 0.5 | 0.025 | 0.475 | 2.09 | 20000 | 3.03 × 10⁻⁵ | 4.13 |
| 21 | RUTHENIUM | | | | | | | |

### Comparative Example 1

The hydrogen generating rate and amount were determined as in Example 1 except that no catalyst was added, and thus obtained data are shown in Table 3.

### Comparative Examples 2 and 3

The hydrogen generating rate and amount were determined as in Example 1 except that the following catalysts were used:

cobalt chloride manufactured by Wako Pure Chemical Industries, Ltd. in Comparative Example 2; and

nickel chloride manufactured by Nakalai Tesque, Inc. in Comparative Example 3.

Thus obtained data are shown in Table 3 together with the amounts of catalysts employed.

TABLE 3

| | | | | HYDROGEN |
|---|---|---|---|---|
| | | | HYDROGEN | GENERATING |
| | | AMOUNT | GENER- | AMOUNT |
| | | OF | ATING | [wt %] |
| | | CATALYST | RATE | (PER 1 g OF |
| | CATALYST | [mg] | [gg⁻¹sec⁻¹] | NaBH₄) |
| COMP. | NONE | 0 | 5.43 × 10⁻⁶ | 1.03 |
| EX. 1 | | | | |
| COMP. | COBALT | 0.05 | 6.78 × 10⁻⁶ | 1.19 |
| EX. 2 | CHLORIDE | | | |
| COMP. | NICKEL | 0.5 | 1.10 × 10⁻⁵ | 1.32 |
| EX. 3 | CHLORIDE | | | |

### Comparative Examples 4 to 11

The hydrogen generating rate and amount were determined as in Example 1 except that the following catalysts were used:

platinum black manufactured by Wako Pure Chemical Industries, Ltd. in Comparative Examples 4 to 6;

nickel particle manufactured by Soekawa Chemical Co., Ltd. in Comparative Example 7;

titanium powder manufactured by High Purity Chemical Laboratory in Comparative Examples 8 and 9; and

ruthenium powder manufactured by High Purity Chemical Laboratory in Comparative Examples 10 and 11.

Thus obtained data are shown in Table 4 together with data concerning the catalysts employed.

TABLE 4

| | | | | | | HYDROGEN |
|---|---|---|---|---|---|---|
| | | AMOUNT | AMOUNT | AVERAGE | HYDROGEN | GENERATING |
| | | OF | OF | PARTICLE SIZE | GENERATING | AMOUNT |
| | | CATALYST | METAL | OF METAL | RATE | [wt %] |
| | CATALYST | [mg] | [mg] | [nm] | [gg⁻¹sec⁻¹] | (PER 1 g OF NaBH₄) |
| COMP. EX. 4 | PLATINUM | 5 | 5 | 14.1 | 1 × 10⁻³ | 8.14 |
| COMP. EX. 5 | PLATINUM | 0.5 | 0.5 | 14.7 | 2.82 × 10⁻⁵ | 5.68 |
| COMP. EX. 6 | PLATINUM | 0.05 | 0.05 | 14.7 | 2.01 × 10⁻⁵ | 2.36 |
| COMP. EX. 7 | NICKEL | 5 | 5 | 800 | 1.47 × 10⁻⁵ | 2.65 |
| COMP. EX. 8 | TITANIUM | 5 | 5 | 100000 | 2.77 × 10⁻⁵ | 2.72 |
| COMP. EX. 9 | TITANIUM | 0.5 | 0.5 | 100000 | 1.65 × 10⁻⁵ | 2.13 |
| COMP. EX. 10 | RUTHENIUM | 0.5 | 0.5 | 100000 | 3.08 × 10⁻⁵ | 3.19 |
| COMP. EX. 11 | RUTHENIUM | 0.05 | 0.05 | 100000 | 1.43 × 10⁻⁵ | 1.54 |

### Comparative Examples 12 to 23

The hydrogen generating rate and amount were determined as in Example 1 except that the following catalysts were used:

titania powder manufactured by Sachtleben Chemie GmbH (UV100) in Comparative Examples 12 and 13;

titania powder manufactured by Degussa AG (P-25) in Comparative Examples 14 to 16;

nickel oxide powder manufactured by Wako Pure Chemical Industries, Ltd. in Comparative Example 17;

titanium oxide powder (TiO) manufactured by Rare Metallics Co., Ltd. in Comparative Example 18;

titanium oxide powder (Ti₂O₃) manufactured by Alfa in Comparative Example 19;

silicon oxide powder manufactured by Fuji Silysia Chemical Ltd. in Comparative Examples 20 to 21;

zeolite manufactured by Tosoh Corp. (Mordenite 30) in Comparative Example 22; and

γ-alumina manufactured by Nikki-Universal Co., Ltd. in Comparative Example 23.

Thus obtained data are shown in Table 5 together with data concerning the catalysts employed.

TABLE 5

| | | | AVERAGE | | HYDROGEN |
|---|---|---|---|---|---|
| | | AMOUNT | PARTICLE | HYDROGEN | GENERATING |
| | | OF | SIZE | GENERATING | AMOUNT |
| | | OXIDE | OF OXIDE | RATE | [wt %] |
| | CATALYST | [mg] | [nm] | [gg⁻¹sec⁻¹] | (PER 1 g OF NaBH₄) |
| Comp. Ex. 12 | TiO₂ | 3.8 | 50 | 2.73 × 10⁻⁵ | 5.82 |
| Comp. Ex. 13 | TiO₂ | 38 | 50 | 4.11 × 10⁻⁵ | 6.31 |
| Comp. Ex. 14 | TiO₂ | 3.8 | 130 | 1.59 × 10⁻⁵ | 4.06 |
| Comp. Ex. 15 | TiO₂ | 0.5 | 130 | 1.31 × 10⁻⁵ | 3.56 |
| Comp. Ex. 16 | TiO₂ | 950 | 130 | 1.18 × 10⁻⁴ | 7.78 |
| Comp. Ex. 17 | NiO | 5 | 4500 | 3.23 × 10⁻⁵ | 2.49 |
| Comp. Ex. 18 | TiO | 5 | 2100 | 3.66 × 10⁻⁵ | 5.02 |
| Comp. Ex. 19 | Ti₂O₃ | 5 | 2700 | 4.71 × 10⁻⁵ | 3.70 |
| Comp. Ex. 20 | SiO₂ | 5 | 3000 | 3.02 × 10⁻⁵ | 8.32 |
| Camp. Ex. 21 | SiO₂ | 33 | 3000 | 4.85 × 10⁻⁵ | 9.88 |
| Comp. Ex. 22 | ZEOLITE | 5 | 200 | 4.63 × 10⁻⁵ | 5.33 |
| Comp. Ex. 23 | γ-Al₂O₃ | 3.8 | 160 | 1.01 × 10⁻⁵ | 1.41 |

### Comparative Examples 24 to 30

The hydrogen generating rate and amount were determined as in Example 1 except that the following catalysts were used:

active carbon (having a specific surface area of 1500 m²/g) manufactured by Cataler Industrial Co., Ltd. in Comparative Example 24;

active carbon (having a specific surface area of 3100 m²/g) manufactured by The Kansai Coke and Chemicals Co., Ltd. (30-SPD) in Comparative Example 25;

active carbon (having a specific surface area of 3224 m²/g) manufactured by Osaka Gas Chemicals (M-30) in Comparative Examples 26 to 28; and

synthetic graphite (having a specific surface area of 1 m²/g) manufactured by Osaka Gas Chemicals (MCMB-25-28) in Comparative Examples 29 and 30.

Thus obtained data are shown in Table 6 together with data concerning the catalysts employed.

TABLE 6

| | | | AVERAGE | | HYDROGEN |
|---|---|---|---|---|---|
| | | AMOUNT OF | PARTICLE | HYDROGEN | GENERATING |
| | | CARBONACEOUS | SIZE OF | GENERATING | AMOUNT |
| | | MATERIAL | CARBONACEOUS | RATE | [wt %] |
| | CATALYST | [mg] | MATERIAL [nm] | [gg⁻¹sec⁻¹] | (PER 1 g OF NaBH₄) |
| Comp. Ex. 24 | ACTIVE | 3.8 | 30000 | 2.36 × 10⁻⁵ | 4.22 |
| | CARBON | | | | |
| Comp. Ex. 25 | ACTIVE | 5 | 30000 | 1.85 × 10⁻⁵ | 5.17 |
| | CARBON | | | | |
| Comp. Ex. 26 | ACTIVE | 5 | 10000 | 1.86 × 10⁻⁵ | 8.81 |
| | CARBON | | | | |
| Comp. Ex. 27 | ACTIVE | 1 | 10000 | 1.22 × 10⁻⁵ | 2.73 |
| | CARBON | | | | |
| Comp. Ex. 28 | ACTIVE | 0.5 | 10000 | 1.02 × 10⁻⁵ | 2.43 |
| | CARBON | | | | |
| Comp. Ex. 29 | SYNTHETIC | 5 | 25000 | 3.45 × 10⁻⁵ | 4.02 |
| | GRAPHITE | | | | |
| Comp. Ex. 30 | SYNTHETIC | 0.5 | 25000 | 1.72 × 10⁻⁵ | 2.01 |
| | GRAPHITE | | | | |

From the results shown in Tables 1 to 6, it has been verified that the hydrogen generating rate and amount are remarkably improved by the catalyst in accordance with the present invention in which a particle of metal oxide, metalloid oxide, or carbonaceous material is caused to carry a fine particle of noble metal as compared with the case using a conventional catalyst. Also, it has been verified that this effect is beyond the extent expectable from the catalytic effect in the case where a particle of metal oxide, metalloid oxide, or carbonaceous material is used alone and the catalytic effect in the case where a fine particle of noble metal is used alone, i.e., it accompanies their synergistic effect. Further, it has been verified that the hydrogen generating rate or amount remarkably improves in particular when the average particle size of noble metal fine particle is less than 1 nm in the catalyst in accordance with the present invention.

### Example 22

Into 33 ml of platinum salt solution (a nitrate solution of platinum having a platinum content of 50 g/l, manufactured by Tanaka Kikinzoku Kogyo K.K.), 100 g of lithium cobaltate powder (LiCoO₂, manufactured by Nippon Chemical Industries Co., Ltd., product name: Cellseed 5, average particle size: 5.9 μm) were immersed, so as to cause the lithium cobaltate powder to carry the nitrate of platinum. Then, the lithium cobaltate powder was held at 250° C. for 5 hours, so as to be dried. Thereafter, the dried powder was fired for 2 hours in air at 450° C., whereby a catalyst in which platinum was carried on lithium cobaltate (having a platinum content of 1.5 wt %) was obtained. Then, using thus obtained catalyst, the hydrogen generating rate and amount were determined as in Example 1, which are shown in Table 7 together with data concerning the catalyst employed.

### Examples 23 and 24

A catalyst in which platinum was carried on lithium manganate or lithium niccolate (having a platinum content of 1.5 wt %) was obtained as in Example 22 except that 100 g of lithium manganate powder (Li_{1.03}Mn_{1.97}O₄, manufactured by Honjo Chemical Corp., average particle size: 26 μm) (Example 23) or 100 g of lithium niccolate powder (LiNi_{0.81}Co_{0.16}Al_{0.03}O₂, manufactured by Sumitomo Metal Mining Co., Ltd., average particle size: 11 μm) (Example 24) were used in place of lithium cobaltate powder. Then, using thus obtained catalyst, the hydrogen generating rate and amount were determined as in Example 1, which are shown in Table 7 together with data concerning the catalyst employed.

### Example 25

Using the catalyst obtained as in Example 2, the hydrogen generating rate and hydrogen generating amount were determined as follows. Namely, after the amount of catalyst (500 mg) shown in Table 7 and 3 g of sodium borohydride were packed into an Erlenmeyer flask having a volume of 100 ml, 1 ml of water was added dropwise thereto at room temperature (about 20° C.) by use of a syringe, and the hydrogen generating rate and amount were determined from the change in level of the volumetric cylinder in a gas analyzer (product code: 6071-4) made by Sibata Scientific Technology, Ltd. Here, the amount of hydrogen generated during the time shown in Table 7 (10 minutes) from the starting of the test was measured, and was defined as a measured value of hydrogen generating amount. The hydrogen generating rate was calculated from the hydrogen generating amount at the lapse of 1 minute after starting the test. Thus obtained data are shown in Table 7 together with data concerning the catalysts employed.

### Examples 26 and 27

A catalyst in which rhodium (1.5 wt %) or ruthenium (1.5 wt %) was carried on lithium cobaltate was obtained as in Example 22 except that 33 ml of a rhodium salt aqueous solution (having a rhodium content of 50 g/l, manufactured by Tanaka Kikinzoku Kogyo K.K.) (Example 26) or a ruthenium nitrate solution (having a ruthenium content of 50 g/l, manufactured by Tanaka Kikinzoku Kogyo K.K.) (Example 27) was used in place of the platinum P salt solution. Using thus obtained catalyst, the hydrogen generating rate and amount were determined as in Example 25, which are shown in Table 7 together with data concerning the catalyst employed.

### Examples 28 and 29

A catalyst in which platinum (1.5 wt %) or rhodium (1.5 wt %) was carried on lithium cobaltate was obtained as in Example 22 in Example 28 except that lithium cobaltate powder (LiCoO₂, manufactured by Nippon Chemical Industries Co., Ltd., product name: Cellseed 20, average particle size: 19.4 μm) was used as the lithium cobaltate powder, or as in Example 26 in Example 29. Using thus obtained catalyst, the hydrogen generating rate and amount were determined as in Example 25, which are shown in Table 7 together with data concerning the catalyst employed.

TABLE 7

| | | | | AMOUNT | | | | |
|---|---|---|---|---|---|---|---|---|
| | | | | OF LITH- | | AVERAGE | | |
| | | | | IUM- | | PARTICLE | | |
| | | | | CON- | | SIZE OF | | |
| | | | | TAIN- | AVERAGE | LITHIUM- | | |
| | | | AMOUNT | ING | PARTICLE | CONTAIN- | | HYDROGEN |
| | | | OF | COMBIN- | SIZE | ING | HYDROGEN | GENERATING |
| | CATALYST | AMOUNT | CARRIED | ED | OF CARRIED | COMBINED | GENERATING | AMOUNT |
| | (CARRIER) + | OF | NOBLE | METAL | NOBLE | METAL | RATE | [wt %] |
| | (CARRIED NOBLE | CATALYST | METAL | OXIDE | METAL | OXIDE | [gg⁻¹ | (PER 1 g OF |
| | METAL) | [mg] | [mg] | [mg] | [nm] | [nm] | sec⁻¹] | NaBH₄) |
| EXAMPLE | LITHIUM COBALTATE + | 3.8 | 0.057 | 3.74 | 2 | 5.9 | 1.58 × 10⁻⁴ | 20(20 min) |
| 22 | PLATINUM | | | | | | | |
| EXAMPLE | LITHIUM | 3.8 | 0.057 | 374 | 2 | 26 | 3.02 × 10⁻⁵ | 6.43(2 hr) |
| 23 | MANGANATE + | | | | | | | |
| | PLATINUM | | | | | | | |
| EXAMPLE | LITHIUM NICCOLATE + | 3.8 | 0.057 | 3.74 | 2 | 11 | 4.87 × 10⁻⁵ | 10.3(2 hr) |
| 24 | PLATINUM | | | | | | | |
| EXAMPLE | LITHIUM COBALTATE + | 500 | 7.5 | 493 | 2 | 5.9 | 3.75 × 10⁻⁴ | 3.4(10 min) |
| 25 | PLATINUM | | | | | | | |
| EXAMPLE | LITHIUM COBALTATE + | 500 | 7.5 | 493 | 2 | 5.9 | 4.57 × 10⁻⁶ | 3.73(10 min) |
| 26 | RHODIUM | | | | | | | |
| EXAMPLE | LITHIUM COBALTATE + | 500 | 7.5 | 493 | 2 | 5.9 | 2.74 × 10⁻⁴ | 4.22(10 min) |
| 27 | RUTHENIUM | | | | | | | |
| EXAMPLE | LITHIUM COBALTATE + | 500 | 7.5 | 493 | 2 | 19.4 | 2.93 × 10⁻⁴ | 3.89(10 min) |
| 28 | PLATINUM | | | | | | | |
| EXAMPLE | LITHIUM COBALTATE + | 500 | 7.5 | 493 | 2 | 19.4 | 1.83 × 10⁻⁴ | 1.93(10 min) |
| 29 | RHODIUM | | | | | | | |

From the results shown in Table 7, it has been verified that the hydrogen generating rate and amount are improved by the catalyst in which a particle of lithium-containing combined metal oxide is caused to carry a fine particle of noble metal.

### Example 30

Into 5 ml of acetone, 500 mg of platinum acetylacetonate were dissolved. The resulting solution was introduced into an autoclave, into which 1 g of ceria-zirconia solid solution powder (manufactured by the method described in Japanese Patent Application Laid-Open No. HEI 9-221304, in which ceria and zironia had a molar ratio of 1:1) and 30 g of dry ice were further added. After being tightly closed, the autoclave was heated and pressurized at a temperature of 150° C. and a pressure of 300 kg/cm², and held for 2 hours, so as to cause the ceria-zirconia solid solution to carry platinum acetylacetonate while in a state where carbon dioxide is a supercritical fluid. Then, the ceria-zirconia solid solution powder was held at 105° C. for 1 hour, so as to be dried. Thus dried product was held in a hydrogen/nitrogen gas flow (comprising 50 ml/min of hydrogen gas and 950 ml/min of nitrogen gas) at 500° C. for 1 hour, so as to be reduced, whereby a catalyst in which platinum was carried on the ceria-zirconia solid solution powder (with a platinum content of 1.3% by weight) was obtained. Using thus obtained catalyst, the hydrogen generating rate and amount were determined as in Example 1, which are shown in Table 8 together with data concerning the catalyst employed.

### Example 31

A catalyst in which platinum was carried on titania-zirconia solid solution powder (having a platinum content of 1.3% by weight) was obtained as in Example 30 except that 1 g of titania-zirconia solid solution powder (manufactured by the following method described in Japanese Patent Application No. HEI 11-068347, in which titania and zironia had a weight ratio of 7:3) was used in place of the ceria-zirconia solid solution powder. Using thus obtained catalyst, the hydrogen generating rate and amount were determined as in Example 1, which are shown in Table 8 together with data concerning the catalyst employed.

(Method of Making Titania-Zirconia Solid Solution Powder)

Into a mixture constituted by 305 g of 28% tetrachlorotitanium solution and 200 g of 18% zirconyl oxynitrate aqueous solution, 1000 g of ion-exchanged water were added, and they were further mixed. The resulting mixed liquid was neutralized with 1456 g of 8% aqueous ammonia solution being added thereto. Thus obtained gel was dried at 150° C., calcinated at 400° C., and further fired at 500° C., whereby titania-zirconia solid solution powder was obtained.

### Example 32

A catalyst in which platinum was carried on titania powder (having a platinum content of 1.3% by weight) was obtained as in Example 30 except that 1 g of titania powder (manufactured by Sachtleben Chemie GmbH, UV100) was used in place of the ceria-zirconia solid solution powder. Using 3.9 mg of thus obtained catalyst, the hydrogen generating rate and amount were determined as in Example 1, which are shown in Table 8 together with data concerning the catalyst employed.

### Examples 33 and 34

The hydrogen generating rate and amount were determined as in Example 30 except that the amount of catalyst employed was changed to 0.5 mg (Example 33), and 0.05 mg (Example 34), which are shown in Table 8 together with data concerning the catalyst employed.

### Examples 35 to 39

Using catalysts obtained as in Examples 30 to 34 except that the reduction processing in the hydrogen/nitrogen gas flow was not performed, the hydrogen generating rate and amount were determined as in Example 1, which are shown in Table 8 together with data concerning the catalysts employed.

TABLE 8

| | CATALYST | | | | | | | | | | HYDROGEN |
|---|---|---|---|---|---|---|---|---|---|---|---|
| | CARRIER | | | CARRIED METAL | | | | | | HYDRO- | GENERAT- |
| | | AVER- | | | AVER- | | | | REDUC- | GEN | ING |
| | | AGE | | | AGE | | AMOUNT | | TION | GENERAT- | AMOUNT |
| | | PARTI- | | | PARTI- | | OF | | PROCESS- | ING | [wt % |
| | | CLE | | | CLE | | CATA- | | ING | RATE | (time)] |
| | | SIZE | AMOUNT | | SIZE | AMOUNT | LYST | CARRYING | (VOL %)/ | [gg⁻¹ | (PER 1 g OF |
| | SPECIES | [nm] | [mg] | SPECIES | [nm] | [mg] | [mg] | METHOD | (VOL %) | sec⁻¹] | NaBH₄) |
| EXAM- | CERIA- | 100 | 3.75 | PLATINUM | 1 OR | 0.05 | 3.80 | SUPER- | HYDRO- | 4.35 × 10⁻⁴ | 20.0 |
| PLE | ZIRCONIA | | | | LESS | | | CRITICAL | GEN(5)/ | | (40 min) |
| 30 | SOLID | | | | | | | METHOD | NITRO- | | |
| | SOLUTION | | | | | | | | GEN(95) | | |
| | | | | | | | | | 500° C., 1 hr | | |
| EXAM- | TITANIA- | 35 | 3.75 | PLATINUM | 1 OR | 0.05 | 3.80 | SUPER- | HYDRO- | 2.05 × 10⁻⁴ | 20.0 |
| PLE | ZIRCONIA | | | | LESS | | | CRITICAL | GEN(5)/ | | (120 min) |
| 31 | SOLID | | | | | | | METHOD | NITRO- | | |
| | SOLUTION | | | | | | | | GEN(95) | | |
| | | | | | | | | | 500° C., 1 hr | | |
| EXAM- | TITANIA | 50 | 3.85 | PLATINUM | 1 OR | 0.05 | 3.90 | SUPER- | HYDRO- | 1.45 × 10⁻⁴ | 20.0 |
| PLE | | | | | LESS | | | CRITICAL | GEN(5)/ | | (90 min) |
| 32 | | | | | | | | METHOD | NITRO- | | |
| | | | | | | | | | GEN(95) | | |
| | | | | | | | | | 500° C., 1 hr | | |
| EXAM- | CERIA- | 100 | 0.494 | PLATINUM | 1 OR | 0.0065 | 0.5 | SUPER- | HYDRO- | 1.88 × 10⁻⁴ | 20.0 |
| PLE | ZIRCONIA | | | | LESS | | | CRITICAL | GEN(5)/ | | (120 min) |
| 33 | SOLID | | | | | | | METHOD | NITRO- | | |
| | SOLUTION | | | | | | | | GEN(95) | | |
| | | | | | | | | | 500° C., 1 hr | | |
| EXAM- | CERIA- | 100 | 0.0494 | PLATINUM | 1 OR | 0.00065 | 0.05 | SUPER- | HYDRO- | 7.35 × 10⁻⁵ | 11.5 |
| PLE | ZIRCONIA | | | | LESS | | | CRITICAL | GEN(5)/ | | (120 min) |
| 34 | SOLID | | | | | | | METHOD | NITRO- | | |
| | SOLUTION | | | | | | | | GEN(95) | | |
| | | | | | | | | | 500° C., 1 hr | | |
| EXAM- | CERIA- | 100 | 3.75 | PLATINUM | 1 OR | 0.05 | 3.80 | SUPER- | NONE | 1.30 × 10⁻⁴ | 10.5 |
| PLE | ZIRCONIA | | | | LESS | | | CRITICAL | | | (120 min) |
| 35 | SOLID | | | | | | | METHOD | | | |
| | SOLUTION | | | | | | | | | | |
| EXAM- | TITANIA- | 35 | 3.75 | PLATINUM | 1 OR | 0.05 | 3.80 | SUPER- | NONE | 1.89 × 10⁻⁴ | 17.8 |
| PLE | ZIRCONIA | | | | LESS | | | CRITICAL | | | (120 min) |
| 36 | SOLID | | | | | | | METHOD | | | |
| | SOLUTION | | | | | | | | | | |
| EXAM- | TITANIA | 50 | 3.85 | PLATINUM | 1 OR | 0.05 | 3.90 | SUPER- | NONE | 1.12 × 10⁻⁴ | 19.7 |
| PLE | | | | | LESS | | | CRITICAL | | | (120 min) |
| 37 | | | | | | | | METHOD | | | |
| EXAM- | CERIA- | 100 | 0.494 | PLATINUM | 1 OR | 0.0065 | 0.5 | SUPER- | NONE | 5.13 × 10⁻⁵ | 10.8 |
| PLE | ZIRCONIA | | | | LESS | | | CRITICAL | | | (120 min) |
| 38 | SOLID | | | | | | | METHOD | | | |
| | SOLUTION | | | | | | | | | | |
| EXAM- | CERIA- | 100 | 0.0494 | PLATINUM | 1 OR | 0.00065 | 0.05 | SUPER- | NONE | 2.96 × 10⁻⁵ | 5.34 |
| PLE | ZIRCONIA | | | | LESS | | | CRITICAL | | | (120 min) |
| 39 | SOLID | | | | | | | METHOD | | | |
| | SOLUTION | | | | | | | | | | |

As can be seen from the results shown in Table 8, it has been verified that the hydrogen generating rate and amount are improved by catalysts in which reduction processing is effected after a particle of metal oxide is caused to carry a fine particle of noble metal under a predetermined pressurizing condition as compared with catalysts which are not subjected to reduction processing.

According to the hydrogen generating method and apparatus of the present invention, as explained in the foregoing, the hydrolysis reaction of a complex metal hydride is remarkably accelerated by a synergistic effect between the catalytic effect of at least one substance selected from the group consisting of metal oxides, metalloid oxides, and carbonaceous materials and the catalytic effect of a noble metal, whereby a sufficient hydrogen generating rate and amount is achieved. Since the catalyst in accordance with the present invention is a water-insoluble solid material, it can be easily isolated and collected so as to be utilized repeatedly, and the amount of catalyst contributing to the reaction can easily be increased and decreased so as to control the hydrogen generating amount.

Therefore, the hydrogen generating method and apparatus of the present invention are quite useful in making it possible to utilize complex metal hydrides as a hydrogen source for fuel cells.

From the invention thus described, it will be obvious that the invention may be varied in many ways. Such variations are not to be regarded as a departure from the spirit and scope of the invention, and all such modifications as would be obvious to one skilled in the art are intended for inclusion within the scope of the following claims.

What is claimed is:

1. A hydrogen generating method for generating hydrogen comprising a step of hydrolyzing a complex metal hydride in the presence of water and a catalyst,
wherein said catalyst comprises a noble metal and at least one substance selected from the group consisting of metal oxides, metalloid oxides, and carbonaceous materials.

2. A hydrogen generating method according to claim 1, wherein both of said substance and noble metal in said catalyst exist such as to be able to come into contact with said complex metal hydride and water in said step.

3. A hydrogen generating method according to claim 1, wherein said complex metal halide is at least one member selected from the group consisting of NaBH₄, NaAlH₄, LiBH₄, LiAlH₄, KBH₄, KAlH₄, Mg(BH₄)₂, Ca(BH₄)₂, Ba(BH₄)₂, Sr(BH₄)₂, and Fe(BH₄)₂;
wherein said metal oxide is at least one metal oxide selected from the group consisting of titanium oxide, nickel oxide, cerium oxide, zeolite, alumina, zirconia, and manganese oxide;
wherein said metalloid oxide is a silicon oxide;
wherein said carbonaceous material is at least one carbonaceous material selected from the group consisting of active carbon, graphite, active char, coke, hard carbon, and soft carbon; and
wherein said noble metal is a platinum group element.

4. A hydrogen generating method according to claim 1, wherein said substance is a particle having an average particle size of 1000 μm or less and said noble metal is a fine particle having an average particle size of 100 nm or less.

5. A hydrogen generating method according to claim 1, wherein said catalyst comprises a lithium-containing combined metal oxide and a noble metal.

6. A hydrogen generating method according to claim 5, wherein said lithium-containing combined metal oxide is a particle having an average particle size of 1000 μm or less and comprising at least one lithium-containing combined metal oxide selected from the group consisting of lithium cobaltate, lithium niccolate, lithium manganate, lithium vanadate, and lithium chromate; and
wherein said noble metal is a fine particle having an average particle size of 100 nm or less and comprising a platinum group element.

7. A hydrogen generating method according to claim 1, wherein said catalyst is one in which said substance is caused to carry said noble metal by use of a highly-heated and highly-pressurized fluid maintained under a pressure of 1.013×10⁶ Pa (10 atm) or higher and at a temperature not lower than the boiling point of said fluid under said pressure.

8. A hydrogen generating method according to claim 7, wherein said highly-heated and highly-pressurized fluid is a supercritical fluid, and wherein said noble metal is a fine particle having an average particle size of 10 nm or less.

9. A hydrogen generating method according to claim 7, wherein said catalyst is one subjected to reduction processing after said substance is caused to carry said noble metal.

10. A hydrogen generating method according to claim 9, wherein said catalyst is one subjected to reduction processing in a reducing gas atmosphere at a temperature of 200 to 800° C. after said substance is caused to carry a fine particle of said noble metal having an average particle size of 5 nm or less.

11. A hydrogen generating apparatus comprising a first container in which a complex metal hydride and water are disposed, a second container in which a catalyst is disposed, and a pipe communicating said first and second containers to each other, said apparatus generating hydrogen by hydrolyzing said complex metal hydride in the presence of water and said catalyst,
wherein said catalyst comprises a noble metal and at least one substance selected from the group consisting of metal oxides, metalloid oxides, and carbonaceous materials.

12. A hydrogen generating apparatus according to claim 11, further comprising a means for supplying said complex metal hydride and water into said second container simultaneously or successively whereby both of said substance and noble metal in said catalyst come into contact with said complex metal hydride and water.

13. A hydrogen generating apparatus according to claim 11, wherein said complex metal halide is at least one member selected from the group consisting of NaBH₄, NaAlH₄, LiBH₄, LiAlH₄, KBH₄, KAlH₄, Mg(BH₄)₂, Ca(BH₄)₂, Ba(BH₄)₂, Sr(BH₄)₂, and Fe(BH₄)₂;
wherein said metal oxide is at least one metal oxide selected from the group consisting of titanium oxide, nickel oxide, cerium oxide, zeolite, alumina, zirconia, and manganese oxide;
wherein said metalloid oxide is a silicon oxide;
wherein said carbonaceous material is at least one carbonaceous material selected from the group consisting of active carbon, graphite, active char, coke, hard carbon, and soft carbon; and
wherein said noble metal is a platinum group element.

14. A hydrogen generating apparatus according to claim 11, wherein said substance is a particle having an average particle size of 1000 μm or less and said noble metal is a fine particle having an average particle size of 100 nm or less.

15. A hydrogen generating apparatus according to claim 11, wherein said catalyst comprises a lithium-containing combined metal oxide and a noble metal.

16. A hydrogen generating apparatus according to claim 15, wherein said lithium-containing combined metal oxide is a particle having an average particle size of 1000 μm or less and comprising at least one lithium-containing combined metal oxide selected from the group consisting of lithium cobaltate, lithium niccolate, lithium manganate, lithium vanadate, and lithium chromate; and
wherein said noble metal is a fine particle having an average particle size of 100 nm or less and comprising a platinum group element.

17. A hydrogen generating apparatus according to claim 11, wherein said catalyst is one in which said substance is caused to carry said noble metal by use of a highly-heated and highly-pressurized fluid maintained under a pressure of 1.013×10⁶ Pa (10 atm) or higher and at a temperature not lower than the boiling point of said fluid under said pressure.

18. A hydrogen generating apparatus according to claim 17, wherein said highly-heated and highly-pressurized fluid is a supercritical fluid, and wherein said noble metal is a fine particle having an average particle size of 10 nm or less.

19. A hydrogen generating apparatus according to claim 17, wherein said catalyst is one subjected to reduction processing after said substance is caused to carry said noble metal.

20. A hydrogen generating apparatus according to claim 19, wherein said catalyst is one subjected to reduction processing in a reducing gas atmosphere at a temperature of 200 to 800° C. after said substance is caused to carry a fine particle of said noble metal having an average particle size of 5 nm or less.
